(19) United States
(12) Patent Application Publication
(10) Pub. No.: US 2002/0037559 A1
(43) Pub. Date: Mar. 28, 2002

(54) PROCESS FOR PRODUCING N-PROTECTED D-PROLINE DERIVATIVES

(76) Inventors: MARTIN SAUTER, VISP (CH); DANIEL VENETZ, NATERS (CH); FABIENNE HENZEN, VISP (CH); DIEGO SCHMIDHALTER, GLIS (CH); GABRIELA PFAFFEN, VISP (CH); OLEG WERBITZKY, VISP (CH)

Correspondence Address: FISHER, CHRISTEN & SABOL, 1725 K STREET N W, SUITE 1401, WASHINGTON, DC, 20006, US

(21) Appl. No.: 09/125,723

(22) PCT Filed: Mar. 12, 1997
(86) PCT No.: PCT/EP97/01262

(30) Foreign Application Priority Data
Mar. 13, 1996 (CH) 656/96

(51) Int. Cl. C12P 13/24

(52) U.S. Cl. 435/107

(57) ABSTRACT

Microorganism which can utilize an N-protected proline derivative of general formula (I) in the form of the racemate or one of its optically active isomers, R¹ meaning —(CH₂)₂—COOH or, optionally substituted in each case, C₁-C₄ alkoxy, aryl or aryloxy, and R² meaning hydrogen or hydroxy, as the only nitrogen, only carbon or only carbon and nitrogen source. These microorganisms can be used in a process for producing N-protected cyclic or aliphatic D-amino acid derivatives of general formulae (II) and (V), A together with —N— and —CH— and R³, R⁴ and R⁵ having the given meanings.

The present invention relates to novel microorganisms which are capable of utilizing an N-protected proline derivative of the general formula

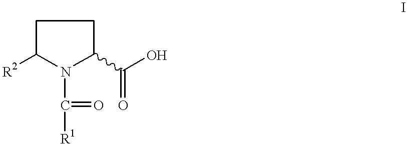

in the form of the racemate or of one of its optically active isomers, in which R¹ is —(CH₂)₂—COOH, in each case optionally substituted C₁₋₄-alkoxy, aryl or aryloxy and R² is hydrogen or ═O, as the sole nitrogen source, as the sole carbon source or as the sole carbon and nitrogen source. These microorganisms and their cell-free enzymes are employed for a novel process for the preparation of N-protected cyclic or aliphatic D-amino acid derivatives and/or of cyclic or aliphatic L-amino acid derivatives.

N-protected cyclic D-amino acid derivatives such as, for example, N-protected D-proline derivatives such as N-benzyloxycarbonyl-D-proline (N-Z-D-proline) are important intermediates for the preparation of pharmaceuticals (J. Org. Chem., 1994, 59, 7496-7498).

As yet, only a few enzymes are known which accept, for example, N-Z-L-proline as a substrate and hydrolyse this to L-proline. These enzymes were isolated from microorganisms of the genus Rhodotorula (JP-A 01 074 987), Pseudomonas (JP-A 55 071 491; Kikuchi et al., Biochim. Biophys. Acta, 744 (1983), 180-188) or from Alcaligenes (JP-A 55 007 015).

All these enzymes react preferably with structurally related substrates of the N-Z-L-proline such as, for example, with N-chloroacetyl-L-proline, but have a low activity with N-Z-L-proline. These enzymes are therefore not suitable for an economical process, for example, for the preparation of N-Z-D-proline. A further disadvantage is that the reaction of the substrate is carried out not with whole cells, but with crude extracts or isolated enzymes, which markedly increases the industrial outlay.

EP-A 0 416 282 discloses an N-acyl-L-proline acylase which prefers, for example, N-acetyl-L-proline as a substrate and is employed for obtaining L-proline. This N-acyl-L-proline acylase is isolated from microorganisms of the species Comamonas testosteroni or Alcaligenes denitrificans. A disadvantage of these microorganisms is that they are not capable of utilizing N-Z-L-proline as the sole nitrogen source and of hydrolysing N-Z-L-proline as a substrate.

WO 95/10604 discloses a microbiological process for the preparation of L-pipecolic acid, by means of microorganisms of the species Alcaligenes denitrificans. These microorganisms also have the disadvantage that they do not utilize the corresponding N-acyl substrate (N-acetyl-(DL)-pipecolic acid) as the sole nitrogen source.

The object of the present invention is to isolate microorganisms which can be employed both for a simple and technically practicable process for the preparation of N-protected cyclic or aliphatic D-amino acid derivatives and for a simple process for the preparation of cyclic or aliphatic L-amino acid derivatives. At the same time, the corresponding products should be isolated in good enantiomeric purity.

This object is achieved by the microorganisms according to claim 1, by the enzymes from these microorganisms according to claim 5 and by the processes according to claims 6, 7, 9 and 10.

The microorganisms according to the invention can be isolated from soil samples, sludge or sewage with the aid of customary microbiological techniques. According to the invention, the isolation of these microorganisms is carried out in such a way that these are cultured in a customary manner in a medium comprising an N-protected proline derivative of the general formula

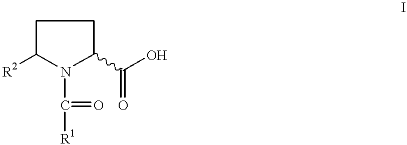

in the form of the racemate or one of its optically active isomers

as the sole carbon and nitrogen source or

as the sole nitrogen source using a suitable carbon source or

as the sole carbon source using a suitable nitrogen source.

From the culture obtained by culturing, those are then expediently selected which utilize an N-protected L-proline derivative of the general formula I as the sole nitrogen source, sole carbon source or sole carbon and nitrogen source.

The radical R¹ in the N-protected proline derivative of the general formula I is —(CH₂)₂—COOH, C₁₋₄-alkoxy, aryl or aryloxy. The radical R² is hydrogen or ═O.

As C₁₋₄-alkoxy, it is possible to use methoxy, fluorenylmethoxy, ethoxy, propoxy, i-propoxy, butoxy, t-butoxy or i-butoxy.

As aryl, a phenyl or benzyl group which is substituted or unsubstituted, such as, for example, 4-methoxybenzyl or 4-methoxyphenyl, is employed.

Aryloxy in the following is defined as a phenyloxy or benzyloxy group, which is substituted or unsubstituted. Examples of an aryloxy group are benzyloxy, 4-methoxy-benzyloxy or 4-nitrobenzyloxy.

The particularly preferred N-protected proline derivatives of the formula I are: N-succinyl-L-proline (R¹=—(CH₂)₂—COOH), N-phenylacetyl-L-proline (R¹=phenylmethyl), N-Z-L-proline (R¹=benzyloxy), N-benzoyl-L-proline (R¹=phenyl), N-isobutoxycarbonyl-L-proline (R¹=isobutoxy) and N-Z-L-pyroglutamate (R¹=benzyloxy, R²=O).

As a suitable carbon source, the microorganisms can utilize, for example, sugars, sugar alcohols or carboxylic acids as a growth substrate. As sugars, hexoses such as, for example, glucose, fructose or pentoses can be used. As carboxylic acids, di- or tricarboxylic acids or their salts can be used, for example citrate or malate. As a sugar alcohol, for example, glycerol can be used.

As a suitable nitrogen source, the microorganisms can utilize, for example, ammonium, nitrate, urea or glycine.

As a selection and growth medium, those customarily used in the specialist field can be used, such as, for example, that described in Table 1. Preferably, that described in Table 1 is used.

During the culture and selection, the active enzymes of the microorganisms are expediently induced. As an enzyme inducer, an N-protected proline derivative of the general formula I or the L-isomer thereof can be used.

Customarily, the culture and selection is carried out at a temperature from 10 to 40° C., preferably from 20 to 35° C. and at a pH between pH 4 and pH 10, preferably between pH 5 and pH 9.

Preferred microorganisms are N-Z-L-proline-utilizing microorganisms of the genus Arthrobacter (first gram-positive microorganism having proline acylase activity), Agrobacterium/Rhizobium, Bacillus, Pseudomonas or Alcaligenes. In particular, microorganisms of the species Arthrobacter sp. HSZ5 having the designation DSM 10328, Agrobacterium/Rhizobium HSZ30, Bacillus simplex K2, Pseudomonas putida K32, Alcaligenes piechaudii K4 or Alcaligenes xylosoxydans ssp. denitrificans HSZ17 having the designation DSM 10329, and also their functionally equivalent variants and mutants, are isolated. The microorganisms DSM 10329 and DSM 10328 were deposited on 6.11.1995 in the Deutsche Sammlung von Mikroorganismen und Zellkultur GmbH, Mascheroderweg 1b, D-38124 Braunschweig, according to the Budapest convention.

“Funtionally equivalent variants and mutants” are understood as meaning microorganisms which essentially have the same properties and functions as the original microorganisms. Variants and mutants of this type can be formed by chance, e.g. by UV irradiation.

Taxonomic description of Alcaligenes xylosoxydans ssp. denitrificans HSZ17 (DSM 10329)

Properties of the strain

| | |
|---|---|
| cell form | rods |
| width μm | 0.5-0.6 |
| length μm | 1.5-3.0 |
| motility | + |
| flaggellation | peritrichous |
| Gram reaction | − |
| lysis by 3% KOH | + |
| aminopeptidase (Cerny) | + |
| spores | − |
| oxidase | + |
| catalase | + |
| anaerobic growth | − |
| ADH (alcohol dehydrogenase) | + |
| NO₂ from NO₃ | + |
| denitrification | + |
| urease | − |
| hydrolysis of | |
| gelatin | − |
| Tween 80 | − |
| acid from (OF test): | |
| aerobic glucose | − |
| xylose 80 | − |
| substrate utilization | |
| glucose | − |
| fructose | − |
| arabinose | − |
| citrate | + |
| malate | + |
| mannitol | − |

Taxonomic Description of Arthrobacter sp. HSZ5 (DSM 10328)

characterization: gram-positive irregular rods having a pronounced rod-cocci growth cycle; strictly aerobic; no acid or gas formation from glucose

| | |
|---|---|
| motility | − |
| spores | − |
| catalase | + |

meso-diaminopimelic acid in the cell wall: no peptidoglycan type: A3α, L-Lys-L-Ser-L-Thr-L-Ala

16S rDNA sequence similarity: sequencing of the range having the greatest variability gave as the highest values 98.2% with Arthrobacter pascens, A. ramosus and A. oxydans

Taxonomic description of Agrobacterium/Rhizobium HSZ30

| | |
|---|---|
| cell form | pleomorphic rods |
| width [μm] | 0.6-1.0 |
| length [μm] | 1.5-3.0 |
| Gram reaction | − |
| lysis by 3% KOH | + |
| aminopeptidase | + |
| spores | − |
| oxidase | + |
| catalase | + |
| motility | + |
| anaerobic growth | − |
| nitrite from nitrate | − |
| denitrification | − |
| urease | + |
| hydrolysis of gelatin | − |
| acid from: | |
| L-arabinose | + |
| galactose | − |
| melezitose | − |
| fucose | + |
| arabitol | − |
| mannitol | − |
| erythritol | − |
| alkalinization of | |
| litmus milk | + |
| ketolactose | − |

The partial sequencing of the 16S rDNA gave comparatively high similarities of about 96% to representatives of the genera Agrobacterium and Rhizobium. Unambiguous assignment to a species described within these genera is not possible.

Taxonomic description of Bacillus simplex K2

| | |
|---|---|
| cell form | rods |
| width [μm] | 0.8-1.0 |
| length [μm] | 3.0-5.0 |
| spores | − |
| ellipsoidal | − |
| circular | − |
| Sporangium | − |
| catalase | + |
| anaerobic growth | − |
| VP reaction | n. g. |
| maximum temperature | |
| growth positive at ° C. | 40 |
| growth negative at ° C. | 45 |
| growth in | |
| medium pH 5.7 | − |
| NaCl 2% | + |
| 5% | − |
| 7% | − |
| 10% | − |
| lysozyme medium | + |
| Acid from (ASS) | |
| D-glucose | + |
| L-arabinose | + |
| D-xylose | − |
| D-mannitol | + |
| D-fructose | + |
| gas from fructose | − |
| lecithinase | − |
| hydrolysis of | |
| starch | + |
| gelatin | + |
| casein | − |
| Tween 80 | + |
| aesculin | − |
| utilization of | |
| citrate | + |
| propionate | − |
| nitrite from nitrate | + |
| indole | − |
| phenylalanine deaminase | − |
| arginine dihydrolase | − |

The analysis of the cellular fatty acids resulted in confirmation of the allocation to the genus Bacillus. The partial sequencing of the 16S rDNA gave a similarity of 100% to Bacillus simplex.

Taxonomic description of Alcaligenes piechaudii K4

| | |
|---|---|
| cell form | rods |
| width [μm] | 0.5-0.6 |
| length [μm] | 1.0-2.5 |
| motility | + |
| flagellation | peritrichous |
| Gram reaction | − |
| lysis by 3% KOH | + |
| aminopeptidase | + |
| spores | − |
| oxidase | + |
| catalase | + |
| ADH | − |
| nitrite from nitrate | + |
| denitrification | − |
| urease | + |
| hydrolysis of gelatin | − |
| substrate utilization | |
| glucose | − |
| fructose | − |
| arabinose | − |
| adipate | + |
| caprate | + |
| citrate | + |
| malate | + |
| mannitol | − |
| pimelate | + |

The profile of the cellular fatty acids is typical of the genus Alcaligenes.

The partial sequencing of the 16S rDNA gave an allocation of 99.3% to the species Alcaligenes piechaudii.

Taxonomic description of Pseudomonas putida K32

| | |
|---|---|
| cell form | rods |
| width [μm] | 0.8-0.9 |
| length [μm] | 1.5-4.0 |
| motility | + |
| flagellation | polar > 1 |
| Gram reaction | − |
| lysis by 3% KOH | + |
| aminopeptidase | + |
| spores | − |
| oxidase | + |
| catalase | + |
| anaerobic growth | − |
| pigments | |
| fluorescent | + |
| pyocyanine | − |
| ADH | + |
| nitrite from nitrate | − |
| denitrification | − |
| urease | − |
| hydrolysis of gelatin | − |
| substrate utilization | |
| adipate | − |
| citrate | + |
| malate | + |
| D-mandelate | + |
| phenylacetate | + |
| D-tartrate | − |
| D-glucose | + |
| trehalose | − |
| mannitol | − |
| benzoyl formate | − |
| propylene glycol | + |
| butylamine | + |
| benzylamine | + |
| tryptamine | − |
| acetamide | + |
| hippurate | + |

The profile of the cellular fatty acids is typical of Pseudomonas putida.

The partial sequencing of the 16S rDNA gave similarities of about 98% to Pseudomonas mendocina and Pseudomonas alcaligenes. The similarity to Pseudomonas putida was 97.4%.

On account of the phenotypic data, this strain, however, can unambiguously be allocated to the species Pseudomonas putida.

The enzymes according to the invention, the N-acyl-L-proline acylases, can be obtained, for example, by customary expert disruption of the described microorganism cells, preferably the enzymes are obtained from Arthrobacter sp. HSZ5 (DSM 10329). For this, for example, the ultrasound, French press or lysozyme method can be used. The enzymes are characterized by the following properties:

N-acyl-L-proline acylase, characterized by the following properties:

a) substrate specificity:

N-benzyloxycarbonyl-L-proline,

N-benzoyl-L-proline,

N-isobutoxycarbonyl-L-proline,

N-benzyloxycarbonyl-L-pyroglutamate,

N-benzyloxycarbonyl-DL-pipecolic acid,

N-benzyloxycarbonyl-L-alanine, are hydrolysed,

b) pH optimum:

the pH optimum is at pH 6.5±0.2

c) temperature stability:

up to 43° C. and pH 6.5 no loss of activity is detectable after incubation for 6 hours.

d) temperature activity:

at 50° C. and pH 6.5 good activity is detectable

e) effects of inhibitors:

benzyl alcohol and N-benzyloxycarbonyl-D-proline have an inhibitory action.

The process according to the invention for the preparation of N-protected cyclic D-amino acid derivatives of the general formula II and/or of a cyclic L-amino acid derivative of the general formula III

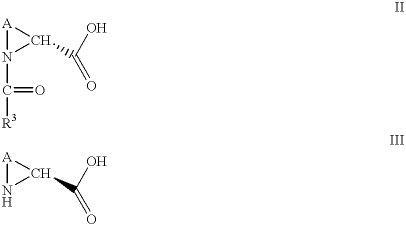

in which A together with —N— and —CH is an optionally substituted 4-, 5- or 6-membered saturated heterocyclic ring and R³ is —(CH₂)₂—COOH, in each case optionally substituted alkyl, alkoxy, aryl or aryloxy, is carried out in such a way that in the racemic N-protected cyclic amino acid derivative of the general formula

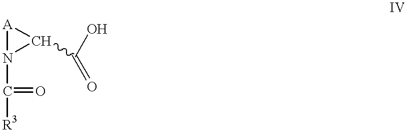

in which A together with —N— and —CH and R³ have the meaning mentioned, the N-protected cyclic L-amino acid derivative is converted by means of the already described microorganisms or by means of their cell-free enzymes into the cyclic L-amino acid derivative (formula III) and this is optionally isolated, where in the biotransformation, in addition to the L-amino acid derivative, the N-protected D-amino acid derivative (formula II), which is optionally isolated, is obtained.

The process for the preparation of N-protected aliphatic D-amino acid derivatives of the general formula V and/or of an aliphatic L-amino acid derivative of the general formula VI

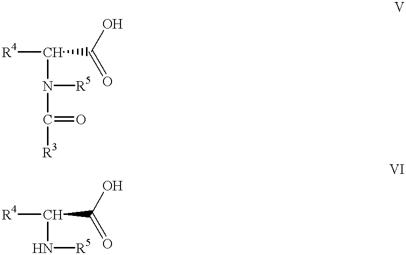

in which R³ has the meaning mentioned, R⁴ is hydrogen, an optionally substituted unbranched alkyl group or an ω-hydroxyalkyl group and R⁵ is hydrogen or an optionally substituted unbranched alkyl group, is carried out analogously to the corresponding cyclic amino acid derivatives. As a starting material for this, a racemic N-protected aliphatic amino acid derivative of the general formula

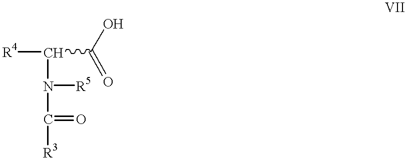

in which R³, R⁴ and R⁵ have the meaning mentioned is employed.

Examples of optionally substituted saturated 5-membered heterocyclic rings are proline, pyrazolidine, imidazoline, oxazolidine, isoxazolidine, thiazolidine and triazolidine. As a substituted saturated 5-membered heterocyclic ring, it is possible to use, for example, 5-oxoproline (pyroglutamate).

Examples of optionally substituted saturated 6-membered heterocyclic rings are piperazine, pipecoline, morpholine, decahydroquinolines, decahydroisoquinolines, quinoxaline. As a 4-membered optionally substituted saturated heterocyclic ring, it is possible to use azetidine.

Alkyl is defined in the following as a C₁₋₁₈-alkyl group, which is substituted or unsubstituted. Examples of a C₁₋₁₈-alkyl group are methyl, chloromethyl, hydroxymethyl, ethyl, propyl, butyl, i-butyl, i-propyl and stearyl. Unbranched alkyl is defined in the following as methyl, ethyl, propyl or butyl. An ω-hydroxyalkyl group is defined in the following as hydroxymethyl, hydroxyethyl, hydroxypropyl or hydroxybutyl.

Alkoxy is defined in the following as a C₁₋₁₈-alkoxy group, which is substituted or unsubstituted. Examples of a C₁₋₁₈-alkoxy group are methoxy, fluorenylmethoxy, ethoxy, propoxy, butoxy, t-butoxy, i-butoxy and stearoxy.

As an optionally substituted aryl or aryloxy group, it is possible to employ the same as those previously described.

Particularly preferred N-protected cyclic or aliphatic amino acid derivatives (starting materials of the formula IV or VII) are: N-Z-proline (R³=benzyloxy), N-t-butoxycarbonylproline (R³=t-butoxy), N-acetylproline (R³=methyl), N-succinylproline (R³=—(CH₂)₂—COOH), N-phenylacetylproline (R³=benzyl), N-benzoylproline (R³=phenyl), N-chloroacetylproline (R³=chloromethyl), N-i-butoxycarbonylproline (R³=—butoxy), N-Z-pipecolinic acid (R³=benzyloxy; 6-membered saturated heterocyclic ring =pipecoline), N-Z-alanine (R³=benzyloxy, R⁴=methyl, R⁵=hydrogen) N-Z-serine (R³=benzyloxy, R⁴ hydroxyethyl, R⁵=hydrogen), N-Z-pyroglutamate (R³=benzyloxy; 5-membered saturated substituted heterocyclic ring=5-oxoproline) and N-Z-sarcosine (R³=benzyloxy, R⁵=methyl).

The preparation of racemic N-protected cyclic or aliphatic amino acids and their derivatives is known in principle. In this preparation, the corresponding L-amino acid is racemized in a known manner according to EP-A 0 057 092, and then reacted in turn in a known manner with the corresponding N-protective group according to Grassmann & Wünsch (Chem. Ber. 91 (1958), 462 - 465).

A process for the preparation of N-protected cyclic or aliphatic amino acids starting from the corresponding L-amino acid in which the racemization and the introduction of the protective group is carried out in an aqueous medium, without isolation of the racemic amino acid, is not known.

In principle, the biotransformation is possible using all microorganisms which utilize an N-protected proline derivative in the form of the racemate or of its optically active isomers as the sole nitrogen source, as the sole carbon source or as the sole carbon and nitrogen source. Likewise suitable are the N-acyl-L-proline acylases isolated from these microorganisms. Particularly suitable for the process are the previously described microorganisms of the genus Arthrobacter, Alcaligenes, Agrobacterium/Rhizobium, Bacillus, Pseudomonas, in particular of the species Agrobacterium/Rhizobium HSZ30, Bacillus simplex K2, Arthrobacter sp. HSZ5, Alcaligenes xylosoxydans ssp. denitrificans HSZ17 (DSM 10329), Pseudomonas putida K32 or Alcaligenes piechaudii K4, and their functionally equivalent variants and mutants.

The biotransformation can be carried out by customary culturing of the microorganisms with resting cells (non-growing cells which no longer need a carbon and energy source) or with growing cells. The biotransformation is preferably carried out with resting cells.

For the biotransformation, technically customary media can be employed, such as, for example, low molarity phosphate buffers, tris buffers, or the medium described in Table 1. The biotransformation is preferably carried out in the medium according to Table 1.

The biotransformation is expediently carried out with single or continuous addition of an N-protected amino acid derivative such that the concentration does not exceed 50% by weight, preferably 20% by weight.

The pH of the medium can be in a range from 3 to 12, preferably from 5 to 9. The biotransformation is expediently carried out at a temperature from 10 to 70° C., preferably from 20 to 50° C.

In the process according to the invention, an N-protected cyclic or aliphatic amino acid derivative is completely converted into a cyclic or aliphatic L-amino acid derivative. In this process, an N-protected D-amino acid derivative is obtained in good yield and enantiomeric purity (ee greater than 98%), and is then isolated.

The N-protected D-amino acid derivative and/or L-amino acid derivative obtained in this manner can be isolated by customary work-up methods such as, for example, by extraction.

### EXAMPLE 1

Selection of N-Z-L-proline-utilizing Microorganisms

A minimal medium (Table 1) was first prepared which fulfilled the growth demands of many microorganisms:

TABLE 1
Minimal medium

| | | |
|---|---|---|
| Na₂SO₄ | 0.1 | g/l |
| Na₂HPO₄.2H₂O | 2.5 | g/l |
| KH₂PO₄ | 1.0 | g/l |
| NaCl | 3.0 | g/l |
| MgCl₂.6H₂O | 0.4 | g/l |
| CaCl₂.2H₂O | 14.5 | mg/l |
| FeCl₃.6H₂O | 0.8 | mg/l |
| trace element solution | 1.0 | ml/l |
| vitamin solution | 1.0 | ml/l |
| pH | 7.0 | |

As a C source, fructose (5 g/1) was added. In order to concentrate microorganisms which are able to hydrolyse N-Z-L-proline selectively, N-Z-L-proline (5 g/l) was added to this base medium as the sole N source. Various batches were then inoculated with soil samples from different locations and incubated (30° C., 120 rpm) until clearly visible growth could be detected. An aliquot of this culture was then inoculated into an equal-size volume of fresh medium and incubated until there was distinct turbidity. This process was repeated three times. The concentrated microorganisms were then isolated and purified on a solid medium (same composition as liquid medium, only addition of 20 g/l of agar-agar). In this manner, approximately 30 different bacterial isolates which were capable of utilizing N-Z-L-proline as the sole N source were obtained.

### EXAMPLE 2

Culture of the Selected Microorganisms

The isolates obtained using the method described in Example 1 were replicated in the medium described there. All cultures which had a sufficient cell density (OD₆₅₀ 2.0) were harvested by centrifugation. The sedimented cells were resuspended and washed in 0.85% NaCl.

After resuspending again in NaCl solution, the ability to hydrolyse N-Z-L-proline was tested using resting cells. For this, a suitable amount of cells was incubated (30° C.) with N-Z-L-proline (5 g/l) in buffer solution (50 mM tris/HCl, pH 7.0). Aliquots were removed at various times and checked for the release of proline from N-Z-proline by means of thin-layer chromatography. A number of isolates exhibited this hydrolytic activity, especially the two strains HSZ5 and HSZ17 identified by the DSM as Arthrobacter sp. and Alcaligenes xylosoxydans spp. denitrificans.

### EXAMPLE 3

Growth and Enzymatic Activity of Arthrobacter sp. HSZ5 (DSM 10328)

Arthrobacter sp. HSZ5 was grown using various C sources (N-Z-L-proline as the N source) or N sources (fructose as the C source). C sources were added to 5 g/l, N sources to 2 g/l. For the induction of the desired enzymatic activity, 1 g/l of N-Z-L-proline, if necessary, was additionally added. Of the C sources tested, only fructose, glucose, sucrose and mannitol were utilized. In all other cases, N-Z-L-proline was used as the C source. The enzymatic activity was dependent only to a slight extent on the C source employed. In contrast to this, all N sources tested were utilized, but the enzymatic activity was in some cases markedly reduced (Table 2):

TABLE 2
Growth and enzymatic activity of Arthrobacter
sp. HSZ5 on culturing with various C (A) or N (B)
sources

| | Cell density | Relative activity |
|---|---|---|
| | [OD₆₅₀] | [%] |
| A) | | |
| C source | | |
| fructose | 12.0 | 100 |
| glucose | 15.0 | 150 |
| sucrose | 12.4 | 148 |
| glycerol | 3.7 | 183 |
| mannitol | 11.2 | 154 |
| citrate | 2.7 | 106 |
| malate | 3.9 | 124 |
| acetate | 3.5 | 144 |
| N-Z-L-proline | 2.8 | 109 |
| B) | | |
| N source | | |
| ammonium | 8.4 | 22 |
| nitrate | 7.6 | 6 |
| urea | 7.8 | 12 |
| glycine | 8.0 | 17 |
| L-glutamate | 9.6 | 43 |
| L-proline | 10.8 | 64 |

### EXAMPLE 4

Inducers of N-acyl-L-proline Acylase

Arthrobacter sp. HSZ5 was grown in minimal medium (Example 1) using fructose (5 g/l) as the C source and L-glutamate (2 g/l) as the N source. N-Z-L-proline, N-Z-DL-proline, N-Z-D-proline, N-Z-sarcosine, N-Z-diethylamine, N-Z-glycine, L-phenylalaninamide, benzamide, N-acetyl-L-proline, N-acetyl glycine, acetamide (in each case 1 g/l) or gelatin (5 g/l) were additionally added. After harvesting the cells, with resting cells the enzymatic activity was in each case tested against N-Z-L-proline and N-Z-D-proline (as described in Example 2). The analytical detection of proline by HPLC showed that only N-Z-L-proline and N-Z-DL-proline induced the desired enzymatic activity, where in both cases only N-Z-L-proline was accepted as the substrate, i.e. the selectivity of the enzyme was high in both cases.

### EXAMPLE 5

Preparation of N-Z-D-proline

a) Arthrobacter sp. HSZ5 was grown in minimal medium (Example 1) using fructose (5 g/l) as the C source and N-Z-L-proline (5 g/l) as the N source. The cells were harvested and washed as described previously. The resting cells (OD₆₅₀=30) were incubated at 30° C. with N-Z-DL-proline (50 g/l) under pH stasis (pH 7.0) and with stirring. At various times, aliquots were taken and the concentration of N-Z-L-proline and N-Z-D-proline was monitored by HPLC (see FIG. 1 +L). After 60 minutes, N-Z-L-proline was almost completely hydrolysed, while N-Z-D-proline was present in the solution unchanged. Thus N-Z-D-proline was present in the solution in high optical purity (ee>99%).

b) Arthrobacter sp. HSZ5 was grown to a cell density of OD₆₅₀>35 at 30° C. in a Chemap fermenter (working volume 2 l) in minimal medium (see Example 1) using glucose (30 g/l) and L-proline (7 g/l) as the C or N source. In order to induce the enzymatic activity, a small amount of N-Z-DL-proline (5 g/l) was then added and the mixture was incubated further for some time. Finally, a further 145 g of N-Z-DL-proline were added continuously over a period of 20 h and the mixture was then incubated for a further 5 hours. The cells were then removed by centrifugation. The fermentation broth was adjusted to a pH of <3 with the aid of hydrochloric acid, and N-Z-proline, which is almost water-insoluble under these conditions, was obtained by extraction with the aid of butyl acetate. An aqueous solution of L-proline and N-Z-proline in organic solvent was obtained by separation of the two phases. The organic phase was concentrated in vacuo and the N-Z-proline obtained was dissolved in ethyl acetate and crystallized by addition of hexane. 41.4 g of N-Z-D-proline whose identity and purity were confirmed by means of ¹H-NMR and melting point determination (75.3° C.) and which had an excellent optical purity (ee>99.5%. according to HPLC, [α]₄=60.0 for c=1 in acetic acid) were isolated here in crystalline form (53.4% of theory).

| | |
|---|---|
| ¹H-NMR (400 MHz in CD₃OD); in ppm: | 7.35 (m, 5H); |
| | 5.1 (m, 2H); |
| | 4.3 (m, 1H); |
| | 3.6-3.4 (m, 2H); |
| | 2.3-2.2 (m, 1H); |
| | 2.1-1.9 (m, 3H) |

c) Arthrobacter sp. HSZ5 was grown to a cell density of OD₆₅₀>30 in a fermenter (nominal volume 20 l) in 6 l of minimal medium (cf. Example 1) using glucose (20 g/l) and L-proline (7 g/l) as the C or N source. For the induction of the enzymatic activity, 112 g of a 50% (w/w) N-Z-DL-proline solution were then added and the mixture was incubated further for 1 h. The volume of the culture was then reduced to 4 l by draining off the unneeded amount. A further 709 g of the 50% (w/w) N-Z-DL-proline solution were then added continuously to these 4 l of culture containing induced cells over a period of 5.5 h and the mixture was then incubated for a further 17.5 h. During the biotransformation, the pH was kept at 7.5-8.5. After completion of the reaction, 4077 g of cell suspension were obtained, from which the cells were removed by ultrafiltration. An aliquot (100 g) of the fermentation broth obtained was worked up as described under 6a). 31.24 g of N-Z-D-proline which with respect to purity (titrimetric content=99.6%) and optical purity (ee>99.5% according to HPLC, [α]⁴=60.2 for c=2 in acetic acid) had excellent values were isolated in crystalline form (85.0% of theory).

d) Arthrobacter sp. HSZ5 was grown to the desired cell density (OD₆₅₀ about 25) at 30° C. in a 450 l fermenter in 250 kg of minimal medium (cf. Example 1) using glucose (13 g/l) and L-proline (7 g/l) as the C or N source. The enzymatic activity was then induced by addition of 4 kg of 50% (w/w) N-Z-DL-proline solution. After an incubation time of 1 h, in which the pH was slowly increased (pH=7.5-8.5), a further 67 kg of the 50% (w/w) N-Z-DL-proline solution were added at a rate of 20 kg/h under pH stasis and the mixture was then incubated for a further 20 h, as a result of the rapid reaction (maximal rate about 12 g of N-Z-L-proline hydrolysed/l×h) the reaction already being almost complete (ee>98%) 8 h after induction. The cells were removed by ultracentrifugation from the 320 kg of culture finally obtained and an aliquot (1444 g) of the cell-free fermentation broth was worked up as described under 6a). 50.0 g of N-Z-D-proline (83.2% of theory) were isolated here in crystalline form in very good purity (titrimetric content>99%) and optical purity (ee>99% according to HPLC).

FIG. 1 +L: Enzymatic hydrolysis of N-Z-L-proline by resting cells of Arthrobacter sp. HSZ5.

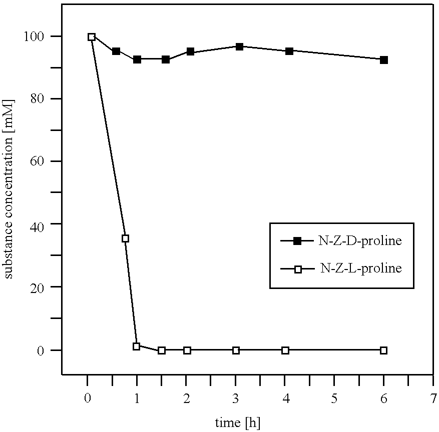

### EXAMPLE 6

Racemization of L-proline

500 mmol of L-proline were dissolved in 125 ml of 4N NaOH (500 mmol) This solution was then heated to 160° C. in a pressure vessel and kept at this temperature for 6 h, a pressure of at most 4.5 bar occuring. After cooling the solution, it was possible by means of the specific rotation ([α]₅=−1.5) to show that proline was present in almost completely racemic form. Similar results were obtained when the racemization was carried out using only 0.15 mol equivalents of NaOH. However, the reaction time had to be increased to 16 h.

### EXAMPLE 7

Preparation of N-Z-(DL)-proline

100.0 g of DL-proline were dissolved in 217 ml of 4N NaOH. Benzyl chloroformate (Z-Cl) was added dropwise to this solution with thermostatting (5-10° C.) and pH stasis (pH=11.5-12.0). Altogether, 158.1 g of Z-Cl and a further 251.2 g of 4N NaOH were added here. 150 ml of distilled water were additionally added in order to maintain the stirrability of the reaction mixture. Using concentrated hydrochloric acid (76 ml), the mixture was then acidified to pH=2.4 and extracted with a total of 584 ml of butyl acetate. N-Z-DL-Proline contained in the organic phase was crystallized analogously to the procedure in Example 6. In this manner, 187.4 g of N-Z-DL-proline (86.5% of theory) were obtained.

### EXAMPLE 8

Preparation of N-Z-(DL)-proline (One-Pot Reaction)

80.0 g of L-proline were dissolved in 174 ml of 4N NaOH and this solution was heated to 160° C. in a pressure vessel. This temperature was maintained for 6 h, a pressure of at most 4.4 bar occuring. After the solution had cooled, the almost complete racemization was determined on the basis of the specific rotation ([α]₅=−0.6). Benzyl chloroformate (Z-Cl) was then added dropwise to this solution with thermostatting (4° C.) and pH stasis (pH=11.5-12.0). In total, 124.5 g of Z-Cl and a further 203.7 g of 4N NaOH were added here, as well as 50 ml of distilled water in order to guarantee the stirrability of the reaction mixture. The mixture was then neutralized by addition of 4.7 g of concentrated hydrochloric acid. After addition of 200 ml of butyl acetate, the pH of the aqueous phase was finally adjusted to 2.0 by addition of 67.4 g of concentrated hydrochloric acid. The aqueous phase was separated off and extracted once more with a total of 200 ml of butyl acetate. The organic phases were combined and N-Z-DL-proline was crystallized analogously to the procedure in Example 6. In this manner, 151.3 g of N-Z-DL-proline (87.3% of theory) were obtained.

### EXAMPLE 9

Characterization of the N-acyl-L-proline Acylase of Arthrobacter sp. HSZ5

a) pH optimum of the N-acyl-L-proline acylase

Arthrobacter sp. HSZ5 was grown (30° C., 120 rpm) in the medium described in Table 1 using fructose (5 g/l) and N-Z-L-proline (5 g/l) as the C or N source. After reaching the desired cell density, the cells were harvested by centrifugation, washed in sodium chloride solution (0.9%) and finally also resuspended in the latter. The enzymatic activity as a function of the pH was determined while maintaining all other parameters (30° C., 50 g/l of N-Z-L-proline, OD₆₅₀=15-20). As the 100% value, the result of the batch at pH=7.0 was employed.

FIG. 2 +L: Activity of the N-acyl-L-proline acylase of Arthrobacter sp. HSZ5 as a function of the pH.

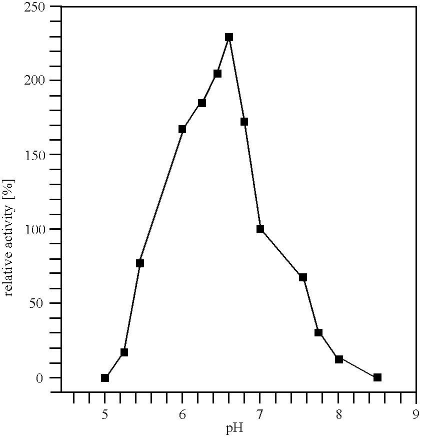

A typical optimum curve resulted here with an optimum in the range of pH=6.4-6.6. At pH=5.0 and pH=8.5, enzymatic activity could no longer be determined.

b) Activity of the N-acyl-L-proline acylase of Arthrobacter sp. HSZ5 as a function of the temperature

Cells having N-acyl-L-proline acylase activity were prepared as described under 9a). This time the enzymatic activity was determined with variation of the temperature. All other parameters were kept constant (pH 6.5, 50 g/l of N-Z-L-proline, OD₆₅₀˜20-25) . As the 100% value, the result of the batch at 30° C. was employed.

FIG. 3 +L: Activity of the N-acyl-L-proline acylase of Arthrobacter sp. HSZ5 as a function of the temperature.

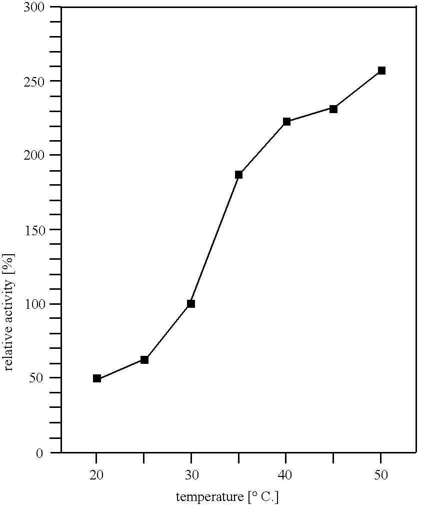

The enzymatic activity increases in the form of a slightly sigmoidal curve. Even at 50° C., good activity is still determinable, which points to good stability of the enzyme.

c) Stability of the N-acyl-L-proline acylase of Arthrobacter sp. HSZ5 as a function of the temperature

Cells having N-Z-L-proline acylase activity were prepared as described under 9a). In order to determine the stability of the enzyme, aliquots of the cell suspension (stirred, pH=6.5, OD₆₅₀˜40-50) were incubated at various temperatures and samples which were analysed under standard conditions (30° C., pH=6.5, 50 g/l of N-Z-L-proline, OD₆₅₀˜15-20) for the remaining activity of the N-acyl-L-proline acylase were taken at different times. The following results were observed here:

up to 43° C. full activity was detectable for at least six hours

at temperatures between 43° C. and 53° C. an increase in the activity even occurred initially, but then a slight loss in activity occurred, such that after five to six hours about 80% of the initial activity was still present

higher temperatures led to the inactivation of the enzyme (50% residual activity after two hours at 60° C. or complete inactivation after one hour at 65° C.)

d) Effect of product inhibition on the activity of the N-acyl-L-proline acylase of Arthrobacter sp. HSZ5 cells having N-acyl-L-proline acylase activity were prepared as described under 9a). Aliquots of the cell suspension were then incubated for 30 minutes (30° C., pH 6.4) with various concentrations of the products obtained in the hydrolysis of N-Z-L-proline (L-proline, N-Z-D-proline, benzyl alcohol), before the enzymatic activity of the respective batches was determined under standard conditions (30° C., pH=6.5, 50 g/l of N-Z-L-proline, OD₆₅₀˜15-20). As the 100% value, the results of the batch was employed here which had been incubated without addition of one of the products.

FIG. 4 +L: Activity of the N-acyl-L-proline acylase of Arthrobacter sp. HSZ5 as a function of the concentration of the products.

[chemical structure]

It is seen here that L-proline also causes no inhibition of the enzymatic activity at all even in high concentration. N-Z-D-proline and benzyl alcohol, on the other hand, lead to a drastic lowering of the enzyme activity with increasing concentration. While N-Z-D-proline appears to act as a competitive inhibitor (linearly increasing inhibition with rising concentration), benzyl alcohol rather inhibits in a non-competitive manner (active above a threshold concentration).

### EXAMPLE 10

Substrate Spectrum of Various Strains With N-acyl-L-proline Acylase

a) Growth using Various N-Protected Amino Acids as the Sole N Source

Arthrobacter sp. HSZ5, Alcaligenes xylosoxidans ssp. denitrificans HSZ17, Agrobacterium/Rhizobium HSZ30, Bacillus simplex K2, Alcaligenes piechaudii K4 and Pseudomonas putida K32 were grown (30° C., 120 rpm) in the medium described in Table 1 using fructose (5 g/l) as the C source. As the sole N source (5 g/l), in each case various N-protected amino acids were added. On reaching a cell density of OD₆₅₀>0.5, the batch was assessed as positive.

TABLE 3
Growth of various strains using various N-
protected amino acids as the sole N source.

| | HSZ5 | HSZ17 | HSZ30 | K2 | K4 | K32 |
|---|---|---|---|---|---|---|
| N-Z-L-proline | + | + | + | + | + | + |
| N-acetyl-L-proline | + | + | + | | | + |
| N-succinyl-L-proline | | | | | | + |
| N-phenylacetyl-L- | + | | | | + | |
| proline | | | | | | |
| N-benzoyl-L-proline | + | | | | | + |
| N-isobutoxycarbonyl- | + | | | | + | + |
| L-proline | | | | | | |
| N-Z-L-pyroglutamate | + | + | + | + | + | + |
| N-Z-L-alanine | + | | + | + | | + |
| N-Z-L-serine | + | | | | | + |
| N-Z-sarcosine | + | | | | | |

b) Enzymatic Hydrolysis of Various N-Protected Amino Acids

Arthrobacter sp. HSZ5, Alcaligenes xylosoxidans ssp. denitrificans HSZ17, Agrobacterium/Rhizobium HSZ30, Bacillus simplex K2, Alcaligenes piechaudii K4 and Pseudomonas putida K32 were grown (30° C., 120 rpm) in the medium described in Table 1 using fructose (5 g/l) and N-Z-L-proline (5 g/l) as the C or N source. After reaching the desired cell density, the cells were harvested by centrifugation and washed in sodium chloride solution (0.9%). After all the strains had been tested for the desired enzymatic activity, the cells were then resuspended in 100 mM potassium phosphate buffer (pH 7.0). After addition of various N-protected amino acids (final concentration 100 mM), the batches were incubated at 30° C. with shaking, samples being removed at suitable intervals. These samples were then analysed for the hydrolysis of the substrates employed.

TABLE 4
Enzymatic hydrolysis of various N-
protected amino acids by whole cells of various N-
acyl-L-proline acylase-containing strains.

| | HSZ5 | HSZ17 | HSZ30 | K2 | K4 | K32 |
|---|---|---|---|---|---|---|
| N-Z-L-proline | + | + | + | + | + | + |
| N-BOC-L-proline | | + | + | | + | + |
| N-acetyl-L-proline | | + | + | | | + |
| N-succinyl-L-proline | | | | | | + |
| N-phenylacetyl-L- | | + | | | + | |
| proline | | | | | | |
| N-benzoyl-L-proline | + | + | + | + | + | + |
| N-chloroacetyl-L- | | + | | | + | |
| proline | | | | | | |
| N-isobutoxycarbonyl- | + | + | + | | + | + |
| L-proline | | | | | | |
| N-Z-DL-pipecolic acid | + | + | | | + | |
| N-Z-L-alanine | + | + | | + | + | |
| N-Z-L-serine | | + | | + | + | |

1. Microorganisms, characterized in that they are capable of utilizing an N-protected proline derivative of the general formula 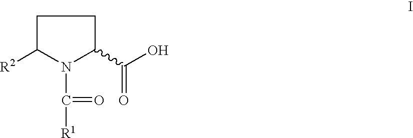
in the form of the racemate or of one of its optically active isomers, in which R¹ is —(CH₂)₂—COOH, in each case optionally substituted C₁₋₄-alkoxy, aryl or aryloxy and R² is hydrogen or ═O, as the sole nitrogen source, as the sole carbon source or as the sole carbon and nitrogen source.

2. Microorganisms according to claim 1, which are capable of utilizing an N-protected L-proline derivative of the general formula I as the sole nitrogen source, sole carbon source or as the sole carbon and nitrogen source.

3. Microorganisms according to claim 1 or 2 of the genus Arthrobacter, Agrobacterium/Rhizobium, Bacillus, Pseudomonas or Alcaligenes.

4. Microorganisms according to at least one of claims 1 to 3 of the species Arthrobacter sp HSZ5 (DSM 10328), Alcaligenes xylosoxidans ssp. denitrificans HSZ17 (DSM 10329), Agrobacterium/Rhizobium HSZ30, Bacillus simplex K2, Pseudomonas putida K32 or Alcaligenes piechaudii K4, and their functionally equivalent variants and mutants.

5. N-Acyl-L-proline acylase, characterized by the following properties:
a) substrate specificity:
N-benzyloxycarbonyl-L-proline,
N-benzoyl-L-proline,
N-isobutoxycarbonyl-L-proline,
N-benzyloxycarbonyl-L-pyroglutamate,
N-benzyloxycarbonyl-DL-pipecolic acid,
N-benzyloxycarbonyl-L-alanine,
are hydrolysed,
b) pH optimum:
the pH optimum is at pH 6.5±0.2
c) temperature stability:
at 43° C. and pH 6.5 no loss in activity is detectable after incubation for 6 hours.
d) temperature activity:
at 50° C. and pH 6.5 good activity is detectable
e) effects of inhibitors:
benzyl alcohol and N-benzyloxycarbonyl-D-proline have an inhibitory action.

6. Process for the preparation of an N-protected cyclic D-amino acid derivative of the general formula II and/or of a cyclic L-amino acid derivative of the general formula III 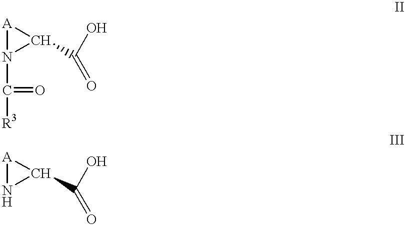
in which A together with —N— and —CH is an optionally substituted 4-, 5- or 6-membered saturated heterocyclic ring and R³ is —(CH₂)₂—COOH, in each case optionally substituted alkyl, alkoxy, aryl or aryloxy, characterized in that in the racemic N-protected cyclic amino acid derivative of the general formula 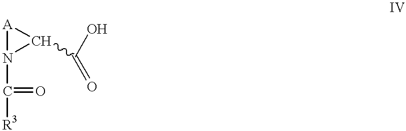
in which A together with —N— and —CH and R³ have the meaning mentioned, the N-protected cyclic L-amino acid derivative is converted by means of the microorganisms according to claims 1 to 4 or by means of the cell-free enzymes according to claim 5 into the cyclic L-amino acid derivative (formula III) and this is optionally isolated, where in the biotransformation, in addition to the cyclic L-amino acid derivative, the N-protected cyclic D-amino acid derivative (formula II) , which is optionally isolated, is obtained.

7. Process for the preparation of an N-protected aliphatic D-amino acid derivative of the general formula V and/or of an aliphatic L-amino acid derivative of the general formula VI 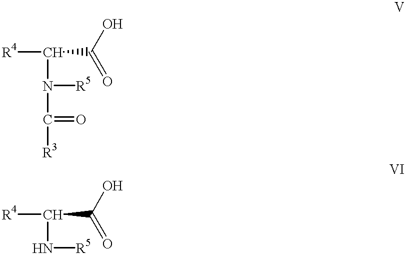
in which R³ has the meaning mentioned, R⁴ is hydrogen, an optionally substituted unbranched alkyl group or an ω-hydroxyalkyl group and R⁵ is hydrogen or an optionally substituted unbranched alkyl group, characterized in that in the racemic N-protected aliphatic amino acid derivative of the general formula 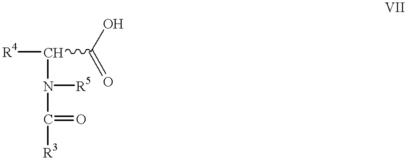
in which R³, R⁴ and R⁵ have the meaning mentioned, the N-protected aliphatic L-amino acid derivative is converted by means of the microorganisms according to claims 1 to 4 or by means of the cell-free enzymes according to claim 5 into the aliphatic L-amino acid derivative (formula VI) and this is optionally isolated, where in the biotransformation, in addition to the aliphatic L-amino acid derivative, the N-protected aliphatic D-amino acid derivative (formula V), which is optionally isolated, is obtained.

8. Process according to claim 6 or 7, characterized in that the biotransformation is carried out by means of microorganisms of the genus Arthrobacter, Agrobacterium/Rhizobium, Bacillus, Pseudomonas or Alcaligenes.

9. Process for the preparation of an N-protected cyclic D-amino acid derivative of the general formula 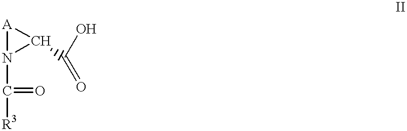
in which A together with —N— and —CH is an optionally substituted 4-, 5- or 6-membered saturated heterocyclic ring and R³ is —(CH₂)₂—COOH, in each case optionally substituted alkyl, alkoxy, aryl or aryloxy, characterized in that a cyclic L-amino acid derivative of the general formula 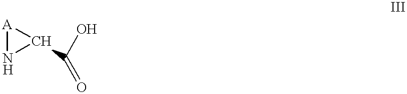
in which A together with —N— and —CH has the meaning mentioned, is racemized to the corresponding cyclic amino acid derivative, this is converted into an N-protected cyclic amino acid derivative of the general formula 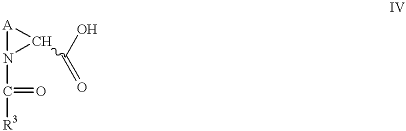
in which A together with —N— and —CH and R³ have the meaning mentioned, and in the latter the N-protected L-amino acid derivative is converted by means of the microorganisms according to claims 1 to 4 or by means of the cell-free enzymes according to claim 5 into the cyclic L-amino acid derivative, this is optionally isolated, where in the biotransformation, in addition to the cyclic L-amino acid derivative, the N-protected cyclic D-amino acid derivative (formula II) , which is isolated, is obtained.

10. Process for the preparation of an N-protected aliphatic D-amino acid derivative of the general formula 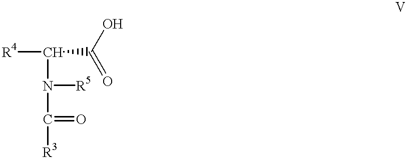
in which R³, R⁴ and R⁵ have the meaning mentioned, characterized in that an aliphatic L-amino acid derivative of the general formula 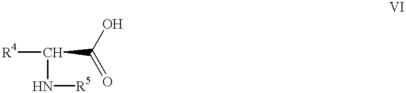
in which R⁴ has the meaning mentioned, is racemized to the corresponding aliphatic amino acid derivative, this is converted into an N-protected aliphatic amino acid derivative of the general formula 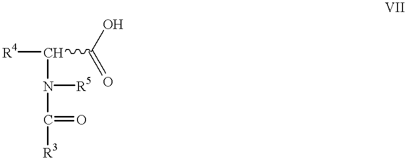
in which R³, R⁴ and R⁵ have the meaning mentioned, and in the latter the N-protected aliphatic L-amino acid derivative is converted by means of the microorganisms according to claims 1 to 4 or by means of the cell-free enzymes according to claim 5 into the aliphatic L-amino acid derivative, this is optionally isolated, where in the biotransformation, in addition to the aliphatic L-amino acid derivative, the N-protected D-amino acid derivative (formula V), which is isolated, is obtained.

11. Process according to claim 9 or 10, characterized in that the reaction is carried out in an aqueous medium without isolation of the racemic amino acid derivative.
